(19) United States
(12) Patent Application Publication
(10) Pub. No.: US 2019/0374720 A1
(43) Pub. Date: Dec. 12, 2019

(54) INJECTION METHOD AND SYSTEM

(71) Applicant: Fady SAID, Jerusalem (IL)

(72) Inventor: Fady SAID, Jerusalem (IL)

(21) Appl. No.: 16/000,919

(22) Filed: Jun. 6, 2018

(51) Int. Cl. A61M 5/30; A61M 5/20; A61M 5/145

(52) U.S. Cl. CPC A61M 5/30; A61M 5/2053; A61C 19/063; A61L 2202/14; A61M 5/145

(57) ABSTRACT

A fluid delivery system includes a fluid delivery apparatus for providing pressurized first fluid and coolant, the fluid delivery apparatus in communication with the nozzle through which the first fluid and coolant leave the system for application at a treatment site.

### TECHNICAL FIELD

The present invention is directed to injection devices for providing various compositions to the body.

### BACKGROUND

Injection devices involve needles to pierce or otherwise penetrate the skin to deliver compositions to the body. This is of particular sensitivity in the oral cavity or the mouth, where needle injections are painful as they must penetrate the skin or gingiva, activating many highly sensitive nerves. Additionally, many people fear needles, especially dental needles, and as a result, avoid dental procedures. In some cases, avoiding dental procedures may be life threatening.

### SUMMARY OF THE INVENTION

The present invention overcomes the problems associated with the pain of injections, by providing an injection device which does not use needles to deliver various compositions to the oral cavity (e.g., mouth) or other target area of the body. Accordingly, the device eliminates a majority of the pain associated with needle injections in the oral cavity. Additionally, as needles are not used, cross-contamination and/or infections associated with needles are eliminated.

The device of the present invention is useful for many medical procedures, such as vaccination, anesthetic or anesthesia delivery, locally and for the entire body, as well as composition delivery to the body, with minimal if any chance for infection from the device.

The present invention is directed to a fluid delivery system for delivering fluids, e.g., gases, liquids, and/or combinations thereof, to a treatment site, in/on an animal, e.g., mammalian, body. The system includes a fluid delivery apparatus for providing pressurized first fluid, such as an anesthetic, and a coolant (coolant fluid). The fluid delivery apparatus is in communication with a nozzle through which the first fluid and coolant leave the system for application at the treatment site.

The present invention is directed to a fluid (e.g., gas, liquid and combinations thereof) delivery system. The system comprises: a fluid delivery apparatus for providing pressurized first fluid; and, a nozzle in communication with the fluid delivery apparatus, the nozzle for receiving the pressurized first fluid from the fluid delivery apparatus, the nozzle including at least one aperture through which pressurized fluid flows out of the nozzle, the at least one aperture dimensioned for further pressurizing the pressurized first fluid to at least a pressure for the first fluid to penetrate tissue at a target site.

Optionally, the fluid delivery system additionally comprises: a coolant delivery apparatus in communication with the nozzle, the coolant delivery apparatus for providing pressurized coolant fluid to the nozzle for delivery to the target site.

Optionally, the nozzle is in communication with the fluid delivery apparatus and the coolant delivery apparatus by at least one conduit.

Optionally, the fluid delivery apparatus includes a pump in communication with the at least one conduit.

Optionally, the fluid delivery system additionally comprises: a source of the first fluid in communication with the pump.

Optionally, the first fluid includes a liquid anesthetic.

Optionally, the coolant delivery apparatus includes a pump in communication with the at least One conduit.

Optionally, the fluid delivery system additionally comprises: a source of the coolant fluid in communication with the pump.

Optionally, the coolant fluid includes liquid.

Optionally, the fluid delivery system additionally comprises a valve in communication with the at least one conduit, and in communication with each of, 1) the fluid delivery apparatus, and, 2) the coolant delivery apparatus.

Optionally, the valve is movable between positions including: a first position where the at least one conduit is closed to fluid flow from each of the fluid delivery apparatus and the coolant delivery apparatus; a second position where the first fluid flows from the fluid delivery apparatus through the at least one conduit; and, a third position where the coolant fluid flows from the coolant delivery apparatus through the at least one conduit.

Optionally, the fluid delivery system additionally comprises a controller in electrical communication with the valve for controlling the valve.

Optionally, the nozzle includes a temperature sensor in electronic communication with the controller.

Optionally, the at least one aperture of the nozzle includes a plurality of apertures.

The invention is also directed to a method for delivering fluid (gases, liquids, and combinations thereof) comprising obtaining a fluid delivery system comprising: a fluid delivery apparatus for providing pressurized first fluid; and, a nozzle in communication with the fluid delivery apparatus, the nozzle for receiving the pressurized first fluid from the fluid delivery apparatus, the nozzle including at least one aperture through which pressurized fluid flows out of the nozzle, the at least one aperture dimensioned for further pressurizing the pressurized first fluid. The nozzle is then moved at least proximate to a target site; and, the first fluid is delivered from the fluid delivery apparatus through the nozzle to the target site at a pressure sufficient for the first fluid to penetrate tissue at the target site.

Optionally, the method additionally comprises, prior to delivering the first fluid from the fluid delivery apparatus, delivering coolant from a coolant delivery apparatus through the nozzle to the target site, at least into contact with the tissue for cooling the tissue.

Optionally, the first fluid and the coolant include liquids.

Optionally, the first fluid includes an anesthetic.

Optionally, the tissue includes at least one of skin and oral tissue.

Unless otherwise defined herein, all technical and/or scientific terms used herein have the same meaning as commonly understood by one of ordinary skill in the art to which the invention pertains. Although methods and materials similar or equivalent to those described herein may be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the present invention are herein described, by way of example only, with reference to the accompanying drawings. With specific reference to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

Attention is now directed to the drawings, where like reference numerals or characters indicate corresponding or like components. In the drawings:

FIG. 1A is a perspective view of an apparatus in accordance with an embodiment of the invention.

FIG. 1B is a rear view of the apparatus of FIG. 1A;

FIG. 2A is a block diagram of the base of the apparatus FIGS. 1A and 1B; and,

FIG. 2B is a block diagram of the head of the apparatus of FIGS. 1A and 1B.

### DETAILED DESCRIPTION OF THE DRAWINGS

In the drawings and in the descriptions that follow, the term “proximal,” as is traditional, shall refer to the end of the instrument that is closer to the user, while the term “distal” shall refer to the end that is farther from the user. Terms referencing orientation, e.g., “top”, “bottom”, “up”, “down”, “left”, “right”, “front”, “rear”, and the like, are used for illustrative purposes with reference to the figures and features shown therein. It is to be understood that embodiments in accordance with the present disclosure may be practiced in any orientation without limitation.

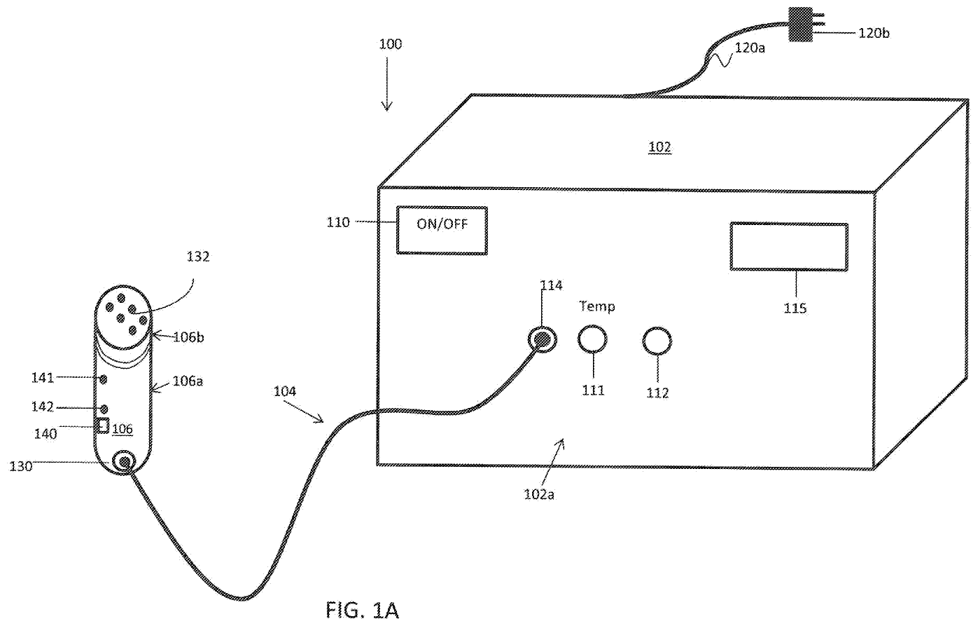

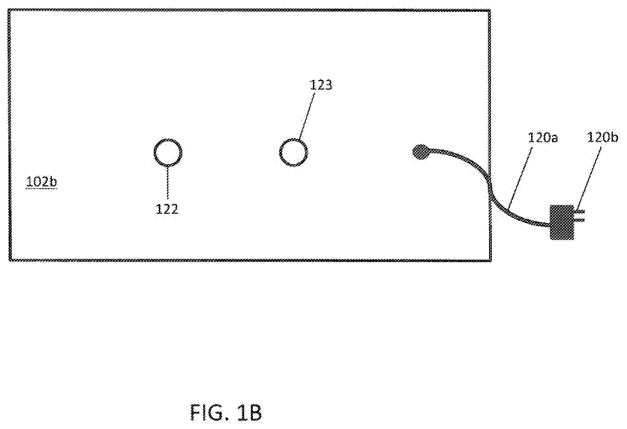

FIGS. 1A and 1B shows the apparatus or device 100 of the present invention. The apparatus 100 is formed of a base 102 in fluid, electronic and data communication, via a line 104, with an operating head 106 (known hereinafter as a “head” or “head member”).

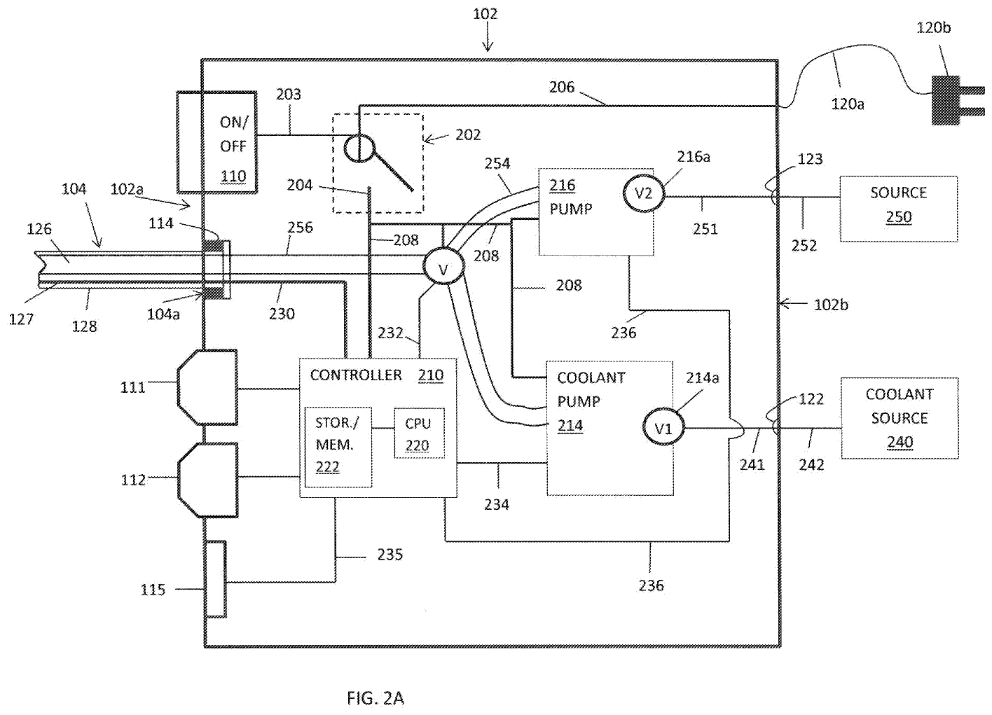

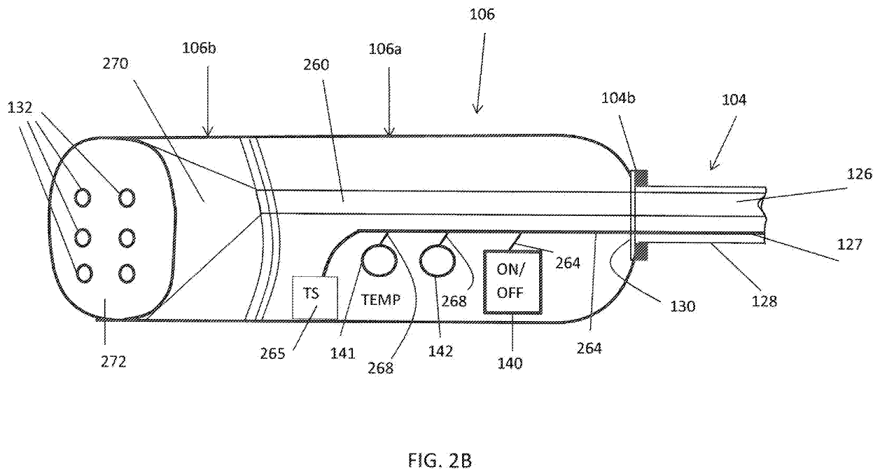

The base 102, for example, on its front side 102a, includes an ON/OFF depressible button 110 for providing power to operate the apparatus 100. There is also a button 111 for activating temperature control, and a button 112 for activating treatment fluid. There is also a port 114 for connecting the line 104 to the outflow line 256 and electrical/data line 230 of the base 102 (FIG. 2A). There is also a display 115 for the temperature at the nozzle 106b.

The rear side 102b of the base 102, as shown in FIG. 1B, includes a cord 120a and plug 120b for connecting with a conventional electric power source (not shown). There are also ports 122, 123. The coolant port 122 serves to connect a coolant line 241 in the base 102 (either directly to a coolant source 240 or via an external line 242, as shown in FIG. 2A. The medical port 123 serves to connect a line 251 in the base 102 (either directly to a treatment fluid source 250 or via an external line 252), as shown in FIG. 2A.

The line 104 supports both fluid and electrical and data connections, and includes connector ends 104a, 1044b which attach to the port 114 of the base 102, and the port 130 of the head 106, with fluid tight connections. The connectors 104a, 104b, for example, connect to the respective ports 114, 130 by male-female frictional connections, luer-lock connections and the like. As shown in FIGS. 2A and 2B the line 104 includes a fluid line 126 and an electrical line 127, covered with and enclosed in a protective sheath 128. The line 104, in particular, the sheath 128, is, for example, of a flexible, waterproof and electrically insulating material, such as a polymer or elastomer. The line 104 is, for example, sterilizable prior to use, or be packaged in a sterile unit.

The head 106 includes a port 130 which receives the line 104 at a line end 104b. The head 106 includes a body 106a and a removably attachable nozzle 106b. The nozzle 106b is shown as being removably attached to the body 106a by a screw fit (cooperative threadings), so as to twist off from the body 106a. Other removably attachable fittings, including friction and other mechanical fittings, between the body 106a and nozzle 106b also permissible.

The nozzle 106b receives fluid, e.g., liquids, gasses, and/or combinations thereof, including coolant fluid and treatment fluid, delivered via the line 104, from a cannula 126 (FIG. 2B), which extends through the body 106a of the head 106. The nozzle 106b terminates in openings or apertures 132. The nozzle 106b is shown with six apertures 132, arranged in two columns of three apertures. However, any arrangement of the apertures 132 and any number of apertures 132 is also permissible, for example, in accordance with the use desired.

The head 106 also includes a power ON/OFF button 140. There is also a button 141 for coolant delivery and control of the rate thereof, and, a button 142 for treatment fluid delivery and control of the rate thereof.

Turning to FIG. 2A, there is shown the base 102 as a block diagram. The ON/OFF button 110 controls a switch 202, via a mechanism 203 e.g., manual, for moving the switch 202 between an open position, as shown, and an engaged position, contact with an electrical lead 204. When the switch 202 is in the engaged position, where there is contact with the electrical lead 204, power from the power source (not shown) is provided through electrical line 206, to electrical lines 208. Electrical lines 208, provide power to the controller 210, a central valve 212, a coolant pump 214, and a pump 216. Alternately, the power source may be a battery, which would be contained within the base (the electrical connections/electrical lines in the base adapted accordingly).

The controller 210 includes a central processing unit 220 and associated storage and memory 222. The controller 210 controls all components of the base 102 and head 106, and all processes performed by the apparatus 100, as detailed herein. This is achieved through electrical and data connection lines from the controller 220 to the head 106, via electrical/data line 230, which connects to electrical/data line 127, from the controller 220 to the central valve 212, via data line 232, and, from the controller 220 to the coolant pump 214, via data line 234. The controller 220 is electrically connected to buttons 111, 112 of the base 102, and is also linked to the screen display 115 via an electrical/data line 235.

The Central Processing Unit (CPU) 220 is formed of one or more processors, including microprocessors, for performing the functions and processes herein, including controlling the components 212, 214, 216, and receiving data from the buttons 140, 141, 142 in the head 106 and the temperature sensor 265 (FIG. 2B) in the head 106. The Central Processing Unit (CPU) 202 processors are, for example, conventional processors, including hardware processors, such as x86 Processors from AMD and Intel, Xenon® and Pentium® processors from Intel, as well as any combinations thereof.

The storage/memory 222 is associated with the CPU 220, includes, for example, machine executable instructions associated with the operation of the above listed components, along with operational processes and subprocesses for the apparatus 100. The storage/memory 204 also, for example, stores rules and policies for the operations and processes of the apparatus 100.

The coolant pump 214 and valve V1 214a are controlled by the controller 210. The coolant pump 214 is connected to a coolant source 240, such as cooled water (at approximately 0 (zero) degrees Celsius to 4 degrees Celsius), liquid coolant, liquefied gas coolants, gas coolants, and the like, via an inflow line 241, which connects to a source line 242, at the port 122. The coolant pump 214 also attaches to an outflow sub line 244, which extends to the central valve 212. As the coolant of the coolant source 240 is fluid, the coolant may be liquid or gas, or combinations thereof. The coolant pump 214 is designed to pump fluid at approximately 0.1 ml/sec to 0.25 ml/sec, The coolant pump 214 may include or be replaced by a pressurizing system, for example, a pneumatic pressurizing system for delivering the coolant.

The pump 216 and valve V2 216a are controlled by the controller 210. The pump 216 is connected to a treatment fluid (liquid, gas or combination thereof) source 250, such as an anesthetic, via an inflow line 251, which connects to a source line 252, at the port 123. The pump 216 also attaches to an outflow sub line 254, which extends to the central valve 212. The pump 216 may include or be replaced by a pressurizing system, for example, a pneumatic pressurizing system for delivering the treatment fluid. The pump 216 is designed to pump fluid, fir example, at approximately 0.2 ml/second, and when coupled with the nozzle 132 at pressures, for example, of approximately 2000 psi to 2600 psi, which is conventionally known to be high pressure.

From the central valve 212, there is an outflow line 256 for the coolant and treatment fluid. This outflow line 256 connects to the fluid line 126 of the line 104, for delivery of coolant and/or treatment fluid to the head 106, for treatment of a body location (treatment site).

Turning also to FIG. 2B, the body 106a includes a fluid line 260, extending the length of the body 106a, from the port 130, where the fluid line 260 connects to line 126, through which coolant and/or treatment fluid are delivered to the head 106 (fir movement therethrough). An electrical/data line 264 extends through the body 106a and connects with the electrical/data line 127 of the line 104, so as to be in electrical and data communication with the controller 210 (via electrical/data line 230).

A temperature sensor and thermometer (TS) 265, for measuring the temperature at the nozzle surface (tip) 272 is in electrical and data communication with the electrical/data line 264. Also, the ON/OFF button for activating the head 106 is in electrical and data communication with the electrical/data line 264, via electrical/data line 266.

The temperature control button 141 is in electrical and data communication with the electrical/data line 264, via electrical/data line 267. By depressing the temperature control button 141, the controller 210 signals the coolant pump 214 to pump more coolant from the coolant source 240 until the desired nozzle surface 272 temperature is reached, as sensed by the temperature sensor 265, with the temperature provided on the screen display 115. The button 141 may be released partially or fully to maintain the desired temperature, at the surface 272 of the nozzle 106b.

The control button 142 is in electrical and data communication with the electrical/data line 264, via electrical/data line 268. By depressing the button 142, the controller 210 signals the pump 216 to pump treatment fluid from the fluid source 250 until the desired amount of treatment fluid has reached the body site. The button 142 may be released partially or fully to maintain the desired fluid flow to the body, through the apertures 132 at the surface 272 of the nozzle 106b.

The nozzle 106b is removably attachable, e.g., separable, from the body 106a, for example, by corresponding threadings on the outside of the nozzle 106b and on the inside of the body 106a. The nozzle 106b includes an outwardly tapered portion 270, with the taper extending outward from the fluid line 260 to the apertures 132. The apertures 132, for example, serve as injectors, providing treatment fluid to the treatment site on the body. Coolant is also received through the line 260, which cools the nozzle 106b, for example, along its outer surface 272, which is designed to be proximate to or contacting the body when in use. The nozzle 106b is, for example, a unitary member, of metal, such as surgical grade steel, or a medical grade plastic which is autoclavable for sterilization. The nozzle 106a can also be of multiple pieces, and can be disposable, for one-time (single) use.

### Example Mode of Operation

The apparatus 100 is designed to deliver treatment fluid, such as anesthetic, to a site (treatment site), such as in the oral cavity (mouth).

In an example operation, the nozzle 106a is initially cooled to a desired temperature, for example, approximately 0 (zero) Celsius (C). This requires the button 141 to be depressed, to communicate to the controller 210, that coolant fluid needs to be delivered to the nozzle 106b. The controller 210 signals the pump 214, to open the valve V1 214a, to draw coolant from the source 240, such that it is pumped, through the line 244 and now-opened central valve V 212 (as opened by the controller 210), through the outflow line 256, and into the line 126 of the line 104, and through the fluid line 260 of the head 106 (all three lines or portions thereof 104, 256, 260 defining one or more conduits), to the nozzle surface 272 (via the tapered portion 270). The button 141 remains depressed until the desired temperature, e.g., 0 (zero) degrees C. is reached and maintained. The head 106, and in particular, the surface 272 of the nozzle 106b may be brought proximal to or into contact with the body site, for treatment, such as spot cooling of tissue. With the button 141 released, the valve V 212 is controlled to move, by the controller 210, such that the paths from lines 244 and 254 to line 256 are closed, until again signaled to open pathways for coolant (depression of button 141) or treatment fluid (depression of button 142).

Next, the button 142 is depressed, and remains depressed, to communicate to the controller 210 that treatment fluid needs to be delivered to the now-cooled nozzle 106b. The controller 210 signals the pump 216, to open the valve V2 216a, to draw treatment fluid, i.e., anesthesia, from the source 250, such that it is pumped, through the line 254 and now-opened central valve V 212 (as opened by the controller 210), through the outflow line 256, and into the line 126 of the line 104, and through the fluid line 260 of the head 106, to the tapered portion 270. The treatment fluid exits the nozzle 106b through the apertures 132, where it is injected into the body at the treatment site. The treatment fluid is delivered at high pressure, for example, approximately 0.2 ml/see, for a short interval, e.g., approximately one second or less, although longer times are suitable depending on the application. The high pressure of delivery allows for the delivery to be over a short time, e.g., one second or less. This small amount of anesthetic, delivered under high pressure for a short time, coupled with the cooled tissue (e.g., spot cooled) penetrates the tissue rapidly (for example, to get under the skin, e.g., epidermal tissue, oral tissue, e.g., gum, or other tissue layer) and numbs the tissue, so that the patient does not feel pain or other sensations during treatment. The button 142 remains depressed until the desired amount of treatment fluid has reached the now cooled treatment site. With the button 142 released, the valve V 212 is controlled to move, by the controller 210, such that the paths from lines 244 and 254 to line 256 are closed, until again signaled to open pathways for coolant (depression of button 141) or treatment fluid (depression of button 142).

Should more of an anesthetic effect be needed, the above-described processes of coolant (coolant fluid) delivery and/or treatment fluid delivery may be repeated as necessary.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof, which would occur to persons skilled in the art upon reading the foregoing description.

1. A fluid delivery system comprising:
a fluid delivery apparatus for providing pressurized first fluid; and,
a nozzle in communication with the fluid delivery apparatus, the nozzle for receiving the pressurized first fluid from the fluid delivery apparatus, the nozzle including at least one aperture through which pressurized fluid flows out of the nozzle, the at least one aperture dimensioned for further pressurizing the pressurized first fluid to at least a pressure for the first fluid to penetrate tissue at a target site.

2. The fluid delivery system of claim 1, additionally comprising:
a coolant delivery apparatus in communication with the nozzle, the coolant delivery apparatus for providing pressurized coolant fluid to the nozzle for delivery to the target site.

3. The fluid delivery system of claim 2, wherein the nozzle is in communication with the fluid delivery apparatus and the coolant delivery apparatus by at least one conduit.

4. The fluid delivery system of claim 3, wherein the fluid delivery apparatus includes a pump in communication with the at least one conduit.

5. The fluid delivery system of claim 4, additionally comprising: a source of the first fluid in communication with the pump.

6. The fluid delivery system of claim 5, wherein the first fluid includes a liquid anesthetic.

7. The fluid delivery system of claim 4, wherein the coolant delivery apparatus includes a pump in communication with the at least one conduit.

8. The fluid delivery system of claim 7, additionally comprising: a source of the coolant fluid in communication with the pump.

9. The fluid delivery system of claim 8, wherein the coolant fluid includes liquid.

10. The fluid delivery system of claim 7, additionally comprising a valve in communication with the at least one conduit, and in communication with each of, 1) the fluid delivery apparatus, and, 2) the coolant delivery apparatus.

11. The fluid delivery system of claim 10, wherein the valve is movable between positions including:
a first position where the at least one conduit is closed to fluid flow from each of the fluid delivery apparatus and the coolant delivery apparatus;
a second position where the first fluid flows from the fluid delivery apparatus through the at least one conduit; and,
a third position where the coolant fluid flows from the coolant delivery apparatus through the at least one conduit.

12. The fluid delivery system of claim 11, additionally comprising a controller in electrical communication with the valve for controlling the valve.

13. The fluid delivery system of claim 12, wherein the nozzle includes a temperature sensor in electronic communication with the controller.

14. The fluid delivery system of claim 12, wherein the at least one aperture of the nozzle includes a plurality of apertures.

15. A method for delivering fluid comprising:
obtaining a fluid delivery system comprising:
a fluid delivery apparatus for providing pressurized first fluid; and,
a nozzle in communication with the fluid delivery apparatus, the nozzle for receiving the pressurized first fluid from the fluid delivery apparatus, the nozzle including at least one aperture through which pressurized fluid flows out of the nozzle, the at least one aperture dimensioned for further pressurizing the pressurized first fluid;
moving the nozzle at least proximate to a target site; and,
delivering the first fluid from the fluid delivery apparatus through the nozzle to the target site at a pressure sufficient for the first fluid to penetrate tissue at the target site.

16. The method of claim 15, additionally comprising, prior to delivering the first fluid from the fluid delivery apparatus, delivering coolant from a coolant delivery apparatus through the nozzle to the target site, at least into contact with the tissue for cooling the tissue.

17. The method of claim 16, wherein the first fluid and the coolant include liquids.

18. The method of claim 17, wherein the first fluid includes an anesthetic.

19. The method of claim 16, wherein the tissue includes at least one of skin and oral tissue.
